(19) United States
(12) Patent Application Publication
(10) Pub. No.: US 2013/0035629 A1
(43) Pub. Date: Feb. 7, 2013

(54) OPTICAL BANDAGE TO STERILIZE WOUNDS

(71) Applicants: Barbara A. Soltz, Spring Valley, NY (US); Robert Soltz, Spring Valley, NY (US)

(72) Inventors: Barbara A. Soltz, Spring Valley, NY (US); Robert Soltz, Spring Valley, NY (US)

(73) Assignee: Conversion Energy Enterprises

(21) Appl. No.: 13/567,985

(22) Filed: Aug. 6, 2012

Related U.S. Application Data
(60) Provisional application No. 61/515,315, filed on Aug. 4, 2011.

(51) Int. Cl. A61M 37/00; A61M 35/00

(52) U.S. Cl. 604/20; 604/304

(57) ABSTRACT

A pad is provided that supplies light to a composite layer, which may be provided on a wound. The composite layer may include a combination including a form of collagen and a chromophore that releases a reactive oxygen species upon exposure to the light. The reactive oxygen species may then act to kill bacteria or other microorganisms in the wound.

This application claims the benefit of provisional application No. 61/515,315 filed Aug. 4, 2011, the entire content of which are incorporated herein by reference.

### BACKGROUND

Many medical disorders are treated with light energy. Tissue may be exposed to light energy to bond, coagulate, debride, sclerose or sterilize it. In addition, tissue may be irradiated with light to stimulate a variety of biological responses including accelerated healing. In addition monochromatic coherent light, monochromatic or polychromatic noncoherent can be therapeutically useful. Applications for exposure span beyond the medical field, with needs for light energy in many areas of industrial manufacturing, food processing, biological research and laboratory testing and analysis. Traditional light delivery systems include optical lenses or fiberoptics which transmit light to provide exposure to a target tissue area. Many medical delivery systems illuminate a circumferential area with a radius of 200 microns to 1 centimeter. Shaped fiber tips can alter the size or shape of the exposed area. Alternatively, light can be delivered to a lens or collimation system to alter the size or shape of delivered energy. All of these devices function in a non-contact mode. Contact fiberoptic tips (e.g. sapphire tips) exist which are heated by light and have a variety of tip contours. The hot tip is then brought in contact with the target tissue to produce the therapeutic effects desired. These existing delivery devices expose a fixed shape (usually circular) to light. Spot sizes vary but they are usually between 0.5 mm and 5 mm.

Areas of tissue that have been wounded are often quite large. Moreover, areas of tissue requiring exposure to light therapy, may be irregular in contour (i.e. mountainous rather than flat). Further, areas of tissue requiring exposure to light may have a contour other than basically flat—i.e. circumferential exposure of tubular structures. When treating such areas it is often difficult to expose the entire area without omitting parts of the area creating “skip zones” that are not adequately treated. This can result in therapeutic failure particularly in the case of light treatment of infected wounds. An excessive amount of time may be required to expose the entire treatment area.

The ability to sterilize open wounds or various types of surgical repairs, especially prior to evidence of infection, is another valuable medical application. Various types of dressings are typically used to seal such surfaces using adhesives or mechanically bandaged in place. These can be mechanically removed or can spontaneously delaminate after a period of time exposing the wounded surface to a series of lethal pathogens. Alternatively, the dressing may be absorbed by the body or hydrated and loosened. The dressings may be impregnated with medications, pharmaceuticals, drugs, growth factors, hormones, enzymes or cells to promote healing, prevent inflammation or thrombosis or other pharmacologic or biological effects. These medications must be re-administered periodically and typically become less effective over time.

### SUMMARY

Consistent with the present disclosure, a delivery system (device, apparatus) is provided for delivering electromagnetic radiation, such as light. The delivery system may include a light activated antimicrobial dressing and a method for using the same. Preferably, the system or device simultaneously exposes an area of target material to a uniform or nearly uniform power density of light. Alternatively, the device may expose an area of target material to a range of power densities which will produce a similar effect or a predictable effect in the target material. In addition, the device may alternatively expose an area of target material to electromagnetic radiation, or coherent monochromatic light or monochromatic light or polychromatic light. Alternatively, the device may deliver or generate electromagnetic radiation for exposure of a diffusing material of the device to generate reactive oxygen ions so that, for example, antimicrobial therapy may be applied to a target tissue.

The device may include several components: 1) an energy transmitting or producing shielding element; 2) a dispersing or diffusing element; 3) a collagen composite mat containing a photosensitizer element, 4) a status sensor element. Some variations of the device may also include: a reflecting element or support.

An energy transmitting element may take energy from a source to which it was coupled and bring this energy to the dispersing or diffusing element, directly to the target material or directly or indirectly to the reflecting element. This component may effect such transportation of energy with a minimum of change in the amount, intensity, and spatial and temporal conformation of the energy. An example of such a transmitting element may include a lens assembly having a collecting or focusing lens. With standard optical coupling to a light emitting device, the lens assembly may transmit the energy, preferably substantially unattenuated, to the dispersing or diffusing element, directly to the target material, or directly or indirectly to the reflecting element.

An energy producing, generating or emitting element may be a component which produces or emits energy which may be, but is not limited to, electromagnetic radiation, polychromatic light, monochromatic non-coherent light, monochromatic coherent light. Examples of this include lasers, diode lasers, light emitting diodes and incandescent bulbs. These may be positioned in such a way as to provide exposure to all or part of the target material or to have an additive effect in conjunction with other cooperatively positioned energy producing elements. Multiple types of energy producing elements may be included for simultaneous or sequential exposure of the target material to produce the desired effects.

The dispersing or diffusing element may be composed of a material which absorbs the input energy at an interface or junction with the transmitting element or energy producing element. This energy is then radiated from the dispersing or diffusing element across its entire surface area, for example, to provide exposure of the target material. This element may be flexible so that it conforms to the target material. In some cases the device may be shaped or formed to produce a cavity, with or without an aperture, which is air filled, the air acting as the dispersing or diffusing element.

The reflecting element typically does not absorb the incident energy but reflects it. The reflecting element may redirect the incident energy, prevent scatter, concentrate the intensity of the energy, and/or redirect or return energy back-scattered, emitted or reflected from the target material or for integrating, smoothing or otherwise modifying the incident energy. Such directing or redirecting may be to or towards the dispersing or diffusing element or a focusing element or the target material. Such integrating or smoothing may produce a more uniform power density, or a non-uniform power density with a specific spatial conformation or a range of power densities with essentially the same or with a predictable or known effect.

A composite material mat may be added on the surface of the diffuser and contacts the tissue. The composite material may be bioresorbable or permanent. This may include material that, when exposed to the incident energy, releases reactive oxygen ions (or reactive oxygen species) that destroy or kill pathogens that cause infection in the tissue. The composition may include a collagen layer with incorporated photosensitive chromophores such as riboflavin, lumichrome or lumiflavin. In another aspect of this disclosure, the collagen composition may be chemically reacted with the chromophore. Once the chromophore is dissolved or incorporated into the collagen composition, the layer or thin film is removed from solution and purified dried to remove any excess chromophore.

A status sensor element that monitors a specific endpoint of exposure may also be provided. This element may alternatively monitor the depletion of the photosensitive chromophore.

The shielding device may be configured as a rectangle, square or other geometric shape. Possible compositions of the shielding component include polyvinyl chloride (PVG), polyethylene (PE), polypropylene (PP), polycarbonate and thermoplastic polyesters, and elastomers and copolymers. Polyvinyl chloride backbone polymers can be formulated with a variety of additives to produce a compound as hard as glass or as soft as gum rubber. To improve tensile strength, resins with higher molecular weights are selected. These polymers are processed by extrusion or injection molding processes. The selection of a plasticizer or processing with elastomers can increase the material flexibility. Autoclaving can be used to change device shape. PVC is a plastic that may be used for short-term, external contact to the body. This material can be extruded into sheets or thin films for additional variation in the conformation of this element of the invention. Four different ethylene based polymer types, with distinct properties, processing characteristics and applications are achievable with varying material density. Typically, the higher the density, the higher the strength, chemical resistance,softening point, stiffness and the lower the optical clarity. In comparison, decreasing density will improve flexibility, impact and tear resistance, sealability and clarity. Therefore, different formulations of this material may be suitable for the shielding and the diffusing element. PVC may be processed by extrusion, injection molding and blow molding methods. In one example, high density polyethylene formulations will be appropriate where a sterile breathable bandage or wound shield is required while low density PE materials will be used as a wound cover, sealant and light trap because of excellent tensile and puncture resistance and flexibility.

Polycarbonate exhibits ductility, dimensional stability, clarity, temperature resistance and biocompatibility. All common molding techniques are currently in use to produce a variety of product shapes. Polycarbonate can be bonded to itself or to other materials by means of a variety of agents and techniques including solvents, adhesives, ultrasonic welding, thermal and vibrational welding. This material may be used as the shielding element especially where there is need to join the shielding element to the diffusing element as part of this invention.

A light source or an array of sources may be assembled with a reflective component that adheres to the inside surface of the rectangle shielding device. The exit face of the rectangle may have affixed to it an additional skirt to prevent leakage of any light from within the exposure chamber. This skirt may be shielding but may also be flexible, stretchable and conformal to the surface of the composite material to insure good seal.

The source of light may be a laser, a diode laser or light emitting diode (LED). As one specific example, one or more emitter/generators may be embedded in the interior (light reflecting) surface of the rectangle. These emitter/generators may produce identical or different power levels. In this case the light device is either mechanically affixed to the shield at a specific point of entry or alternatively can become an integral part of the shield body. The latter example is particularly feasible for semiconductor light emitting diodes. Each diode is embedded into the polymeric material at some processing step such as in the molding process. The diodes may be positioned exactly such that each emitting area is projecting light on the underside of the shield and toward the diffuser element surface. The electrical leads for powering the diodes will also be encapsulated but will have terminal connectors for external connection to a power supply or battery. Further control of the emitting light can be accomplished by including optical elements such as collimating and focusing lens assemblies. The focal length of the focusing lens can be selected to vary spot size (power density) and distance to collagen composite surface. These elements could be incorporated with the diode at the molding processing step of the plastic shield element.

The shape of the shielding device need not be rectangular. It may be alternatively be hemicylindrical, irregular or another geometric shape depending on the size and shape of the area to be exposed depending on the application as well as the desired distribution of energy over that area. It is sometimes necessary to achieve specific therapeutic, manufacturing or processing ends to expose the composite material to a non-uniform density of energy. For example scattering of light within a tissue tends to cause deposit of a higher overall amount of energy in the center of the composite material. It therefore could be necessary for uniform tissue effect to increase the amount of exposure at the periphery of the target site. Other variations in distribution are necessary because of variations in tissue contour, type and other factors. Shape of the reflecting surface or position, intensity or number of light emitters could produce the necessary variation in exposure powers.

Consistent with a further aspect of the present disclosure, the collagen composite layer may be formulated to produce a flexible conformal mat which could be draped over or wrapped around the wound. An outer layer of shielding material would cover the mat. This would be pierced at one or more locations by a transmitting element. Alternatively, energy generating or emitting elements could be embedded on the undersurface (exposure surface) of the mat. These energy generating or emitting elements could be powered by an external or internal power supply. An external power supply could be transmitted by wire or by induction of current in transforming coils within the generating element. Internal power supplies could include batteries and/or capacitors.

The undersurface of the dispersing medium would be in contact with the collagen composite material. This material could include collagen, derivatized collagen, gelatin and a mixture of collagen and gelatin. The material could be imbued with riboflavin, lumichrome, lumiflavin and medications.

The conformation of the mat can be variable. It can be oval rectangular, square or round. It can have single or multiple energy inputs or emitters/generators. It can be spatulate and ribbon-like. It can be cylindrical shaped or produce an inner lumen. The cylinder can have a slit along its length to allow application around circumferential structures. The size and thickness of the device can vary depending on requirements of the applications and the power density/distribution desired.

Consistent with an additional aspect of the present disclosure, a layer may be provided to treat infected tissue or a wound which comprises a composition of collagen and a chromophore, such as riboflavin, lumichrome, lumiflavin, as well as combinations including two or more of the foregoing, and applying that layer on a surface of the infected tissue or wound. The invention also includes directing a beam of photoradiation toward the collagen composition that includes riboflavin provided on the surface of the tissue or wound such that the beam exposes the composition to the photoradiation, the photoradiation including light having a wavelength in a range of 360-375 nm or 440-480 nm with power densities ranging from 0.1 W/cm² to 2 W/cm². In one example, the radiation is light having a wavelength of 455 nm.

The sensor may measure pressure, strain, flow of the collagen composition, and/or the property of light (e.g., wavelength or intensity) reflected, emitted, and/or absorbed by the collagen composition. The sensor may be incorporated within the light shield. Electronics or control circuitry may be included on the control unit circuit card and may be electrically connected to the sensor via a cable. Once the photosensitizer has been depleted, the sensor signal may output change or signal indicating that the exposure is completed.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate one (several) embodiment(s) of the invention and together with the description, serve to explain the principles of the invention.

FIG. 1 illustrates a perspective view of an apparatus including a pad consistent with the present disclosure;

FIG. 2 illustrates a cross-sectional view of the pad shown in FIG. 1;

FIG. 3 illustrates a plan view of the pad shown in FIG. 1;

FIG. 4 illustrates one side of a flexible substrate to be included in a pad consistent with an aspect of the present disclosure;

FIG. 5 illustrates another side of the flexible substrate shown in FIG. 4;

FIG. 6 illustrates a cross-sectional view of the flexible substrate shown in FIG. 4;

FIG. 7 illustrates an example of an apparatus consistent with the present disclosure having a remote power supply;

FIG. 8 illustrates an example of an apparatus consistent with the present disclosure having optical sources;

FIG. 9a is a plan view showing the pad illustrated in FIG. 8 in greater detail;

FIG. 9b is a cross-sectional view of the pad shown in FIG. 8; and

FIGS. 10a, 10b, 11, and 12 illustrate examples of the operation of exemplary pads consistent with the present disclosure.

### DETAILED DESCRIPTION

Reference will now be made in detail to the present exemplary embodiments, which are illustrated in the accompanying drawings. Wherever possible, the same reference numbers will be used throughout the drawings to refer to the same or like parts.

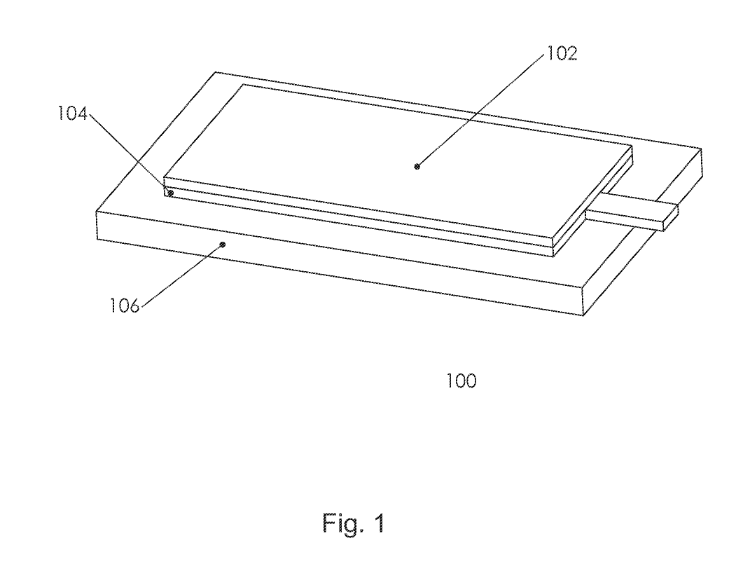

FIG. 1 illustrates an example of an apparatus 100 consistent with an aspect of the present disclosure. Apparatus 100 includes a pad 106, upon which a composite layer 102 is provided. In one example, an intermediate layer 104, including Tegaderm® (commercially available from 3M Corp., St. Paul, Minn.) may be provided between composite layer 102 and pad 106, such that composite layer 102 is provided on surface 104-1 of intermediate layer 104. Put another way, intermediate layer 104 is provided on side 102-1 of composite layer 102. Composite layer 102 may include, for example, collagen and a chromophore. The chromophore may produce a reactive ion oxygen species upon exposure to light. In one example, a concentration of the collagen in the composite layer 102 is 100 mg/ml (10%) up to 600 mg/ml (60%). The collagen may be derivatized with a COO⁻ functional group, and the chromophore includes riboflavin. Exemplary collagen compositions are described in U.S. Pat. Nos. 6,773,699, 6,780,840, and 6,939,364, the entire contents of each of which are incorporated herein by reference. In addition, collagen compositions are disclosed in U.S. Patent Application Publication No. 2011/0125187, the entire contents of which are incorporated herein by reference. The chromophore may also include lumichrome or lumiflavin. In addition, the chromophore may include a combination or two or more of riboflavin, lumichrome, and lumiflavin.

Consistent with an additional aspect of the present disclosure, a concentration of the riboflavin in the composite layer 102 is within a range of 0.1% to 2.0% (w/w). Consistent with a further aspect of the present disclosure, the concentration of the riboflavin in the composite layer is substantially equal to 1.0%. Further, the chromophore may include lumichrome, and the collagen may be gelatinized.

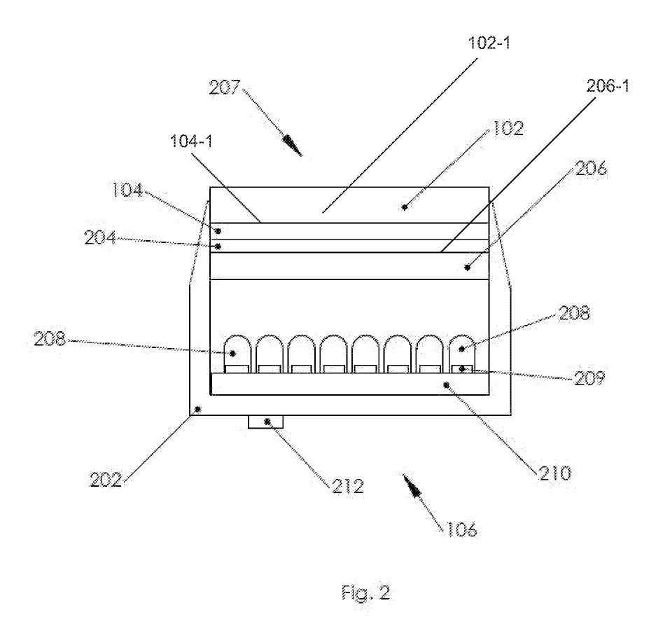

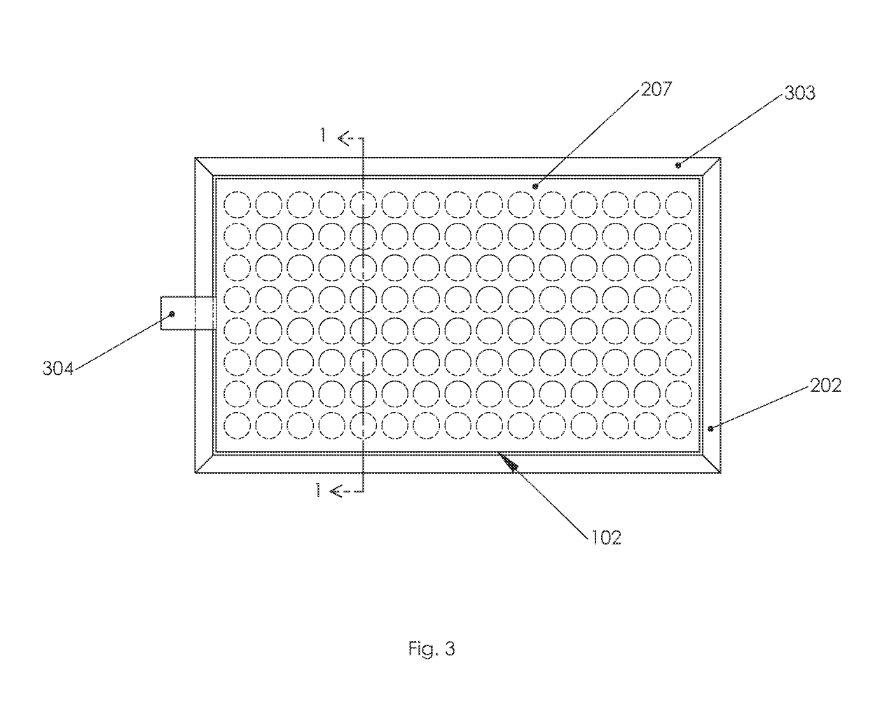

FIG. 2 illustrates a cross-sectional view of pad 106 taken along section 1-1 shown in FIG. 3. Pad 106 may include a housing or frame 202 enclosing a backing or reflective portion 210 (also referred to herein as a “reflector”). A plurality of sources 208 of electromagnetic radiation may be provided on reflector 210, which reflects such electromagnetic radiation toward composite layer 102, when applied to a wound, for example. Sources 208 may be activated to emit the electromagnetic radiation by a switch 212 in order to supply an electrical voltage or current via traces 209 to sources 208. Sources 208 may include light emitting diodes (LEDs), lasers or other appropriate sources of electromagnetic radiation. The electromagnetic radiation, which may include light having a wavelength in a range of 440 nm to 480 nm, for example, may pass through optical diffuser 206, which diffuses the electromagnetic radiation or light output from sources 208. In one example, the wavelength is 450 nm. Optical diffuser 206 may include glass, acrylic, or Teflon®, for example. Preferably, optical diffuser 206 is configured such that composite layer 102 is uniformly exposed by light emitted from sources 208.

The diffused light may next pass through an optional plastic sheet 204, which includes a material that is substantially transparent to the light emitted by sources 208.

As further shown in FIG. 2, intermediate layer 104 may be provided on and in contact with surface 204-1 of plastic sheet 204. Alternatively, intermediate layer 104 may be provided on and in contact with surface 206-1 of diffuser 206. Intermediate layer 104 may include polyacetic acid or polyvinyl alcohol. Optionally, intermediate layer 104 and/or plastic sheet 204 may include Tegaderm® or a plastic polymer. Either surface 104-1, 204-1, or 206-1 may be considered a surface of pad 106.

In one example, composite layer 102 may be provided on intermediate layer 104, as noted above. In particular, in the example shown in FIG. 2, the composite layer 102 is provided in opening 207 of frame 202.

Alternatively, composite layer 102 and intermediate layer 104, including Tegaderm®, may be provided separate from the rest of the components of pad 106 shown in FIG. 2. In this example, composite layer 102 and intermediate layer 104 may be attached, bonded or laminated to one another and the composite layer portion of such laminate may be placed in contact with a wound. Put another way, Diffuser 106 or plastic sheet 204 (if present) may then be brought into contact with intermediate layer 104 along with sources 202 and the other components of pad 106 shown in FIG. 2. The sources may then be activated to emit electromagnetic radiation to expose composite layer 102, as discussed in greater detail below.

FIG. 3 illustrates a plan view of pad or apparatus 100 including composite layer 102. Here, composite layer 102 includes a tab 304, which can be grasped by a user in order to peel away composite layer 102 from intermediate layer 104. As further shown in FIG. 3 apparatus 100 includes an array of sources 208, such as light emitting diodes (LEDs) that emit light in the range of 440 nm to 480 nm. In one example, a portion 303 of frame 202 may have a beveled shape in order to ease application of composite layer 102 to a wound.

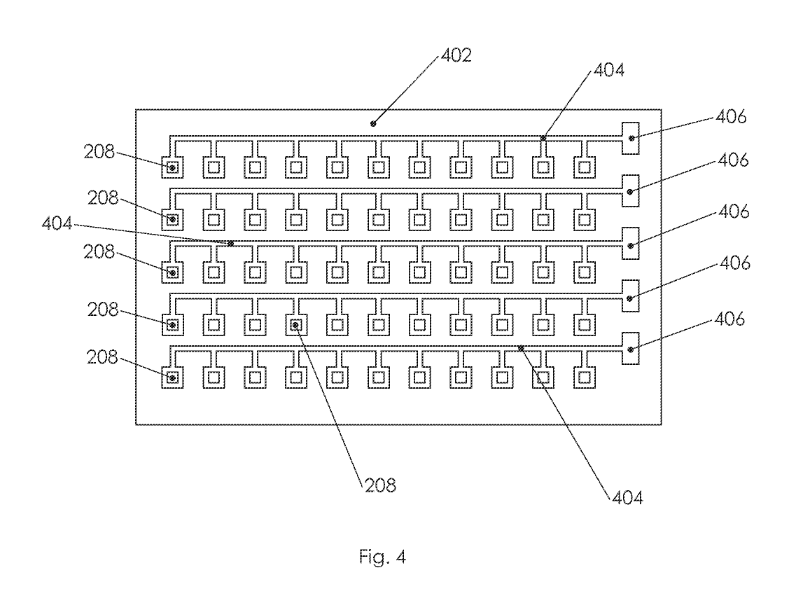

FIG. 4 illustrates portion 400 of apparatus 100 upon which sources 208 may be provided. Portion 400 includes a flexible circuit substrate 402, for example. Wiring or conductive traces or layers 404 (shown as traces 207 in FIG. 2) may be provided to supply a current or voltage from conductive pads 406 to power or activate sources 208.

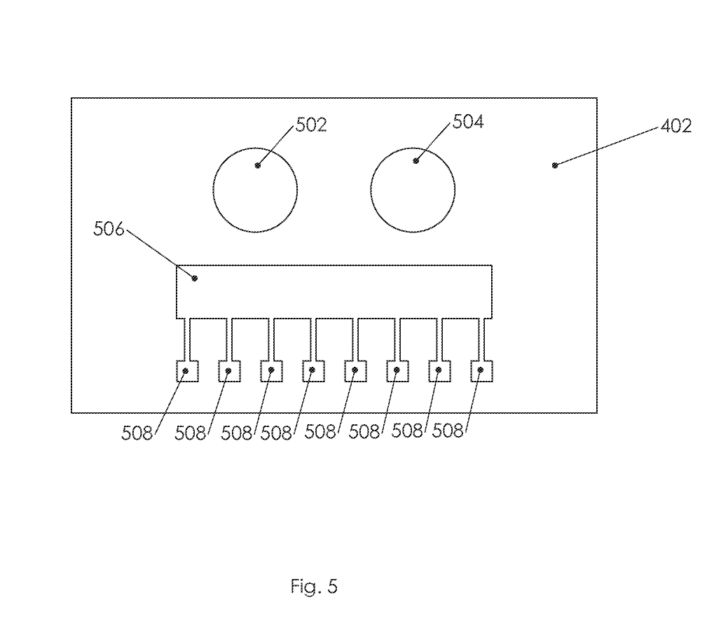

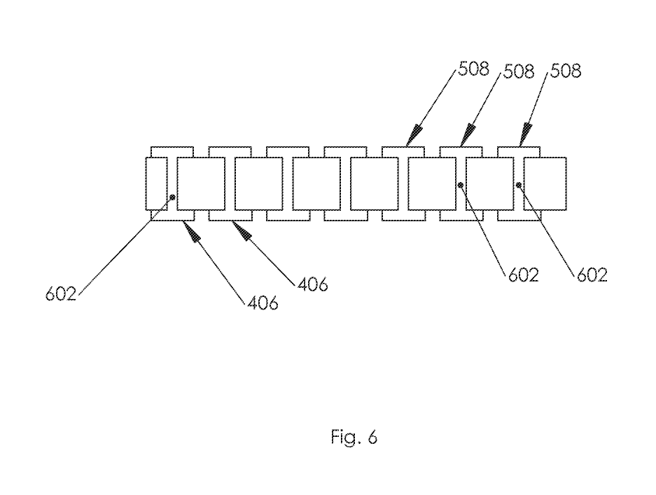

FIG. 5 illustrates an opposite side of substrate 402 than that shown in FIG. 4. In this example, batteries 502 and 504 may be provide on substrate 402, to supply electrical power, in the form of a voltage or current for example, to a control circuit 506. Control circuit 506, in turn, may generate or direct electrical power (current and/or voltage) to conductive pads 508 to appropriately drive each of sources 208 to emit a desired amount of light or optical power. Each of conductive pads 508 is electrically connected to a corresponding one of conductive pads 406, as shown in greater detail in FIG. 6, which is a cross-sectional view of substrate 402. For example, each of pads 508 is electrically connected to a corresponding one of pads 406 via a conductor 602 that extends through a corresponding one of vias 603 provided in substrate 402.

It is noted that the present disclosure is not limited to the number of batteries shown in 5. Rather any appropriate number of batteries may be provided based on the electrical power requirements of sources 208 and current and/or voltage ratings of the batteries or battery.

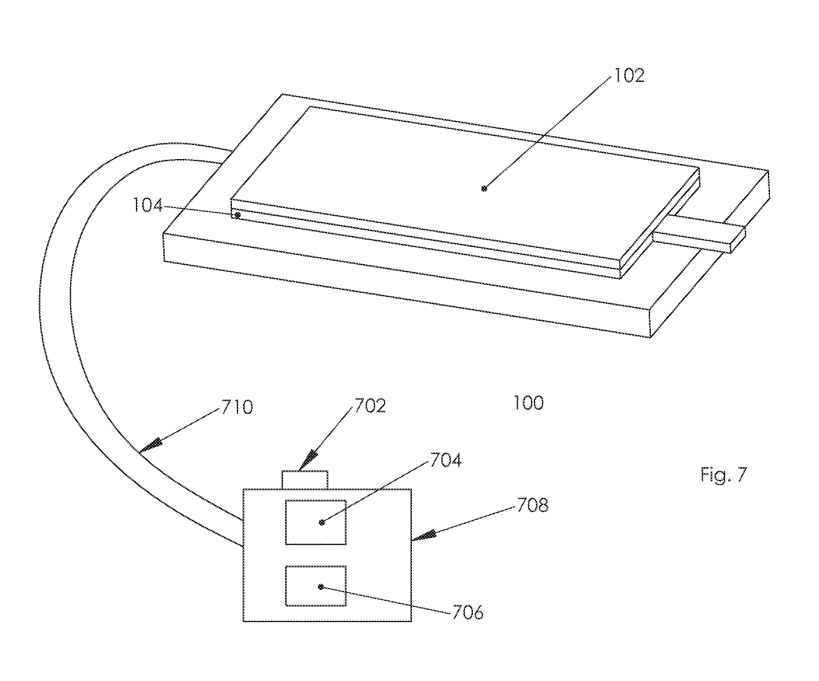

In the examples discussed above, the power supply (e.g., batteries) and control circuitry 506 is housed within frame 202 of pad 106. Consistent with an additional aspect of the present disclosure, however, the power supply, e.g. battery or batteries may be provided remote from the apparatus 100. For example, as shown in FIG. 7, one or more batteries 704 or other power supplies may be provided in housing 708. Control circuitry 706, similar to control circuit 506 discussed above, may also be provided in housing 708, such that upon, activation by switch 702 by a user, for example, an appropriate drive signal (voltage and/or current) may be output from housing 708 via conductive cable or wire 710 to pad 106. Cable 710 may be connected to one or more traces 404, so that the desired drive signal may be applied to activate sources 208 to emit light. Control circuitry 708 may also regulate the current and/or voltage output sources 208.

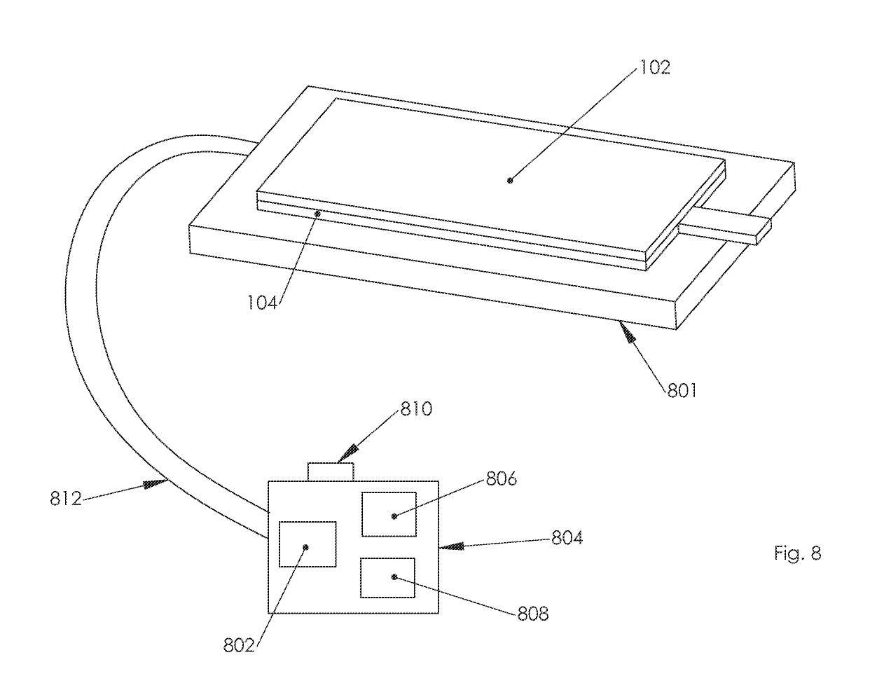

FIG. 8 illustrates an alternative embodiment 800 in which one or more sources 202 discussed above are omitted. Instead, in this example, one or more light or optical sources 802 are provided remote from apparatus or pad 801 in housing 804. In addition, a power supply 806 (including one or more batteries, for example) and control circuit 808 are provided in housing 804. Upon activation by switch 810, control circuit 808, in conjunction with power supply 806, activates one or more of optical sources 802 to emit light at a desired intensity or power. Sources 802 may include one or more lasers or one or more LEDs that output the light to an optical waveguide, such as a known optical fiber 812, which directs the light to apparatus 801.

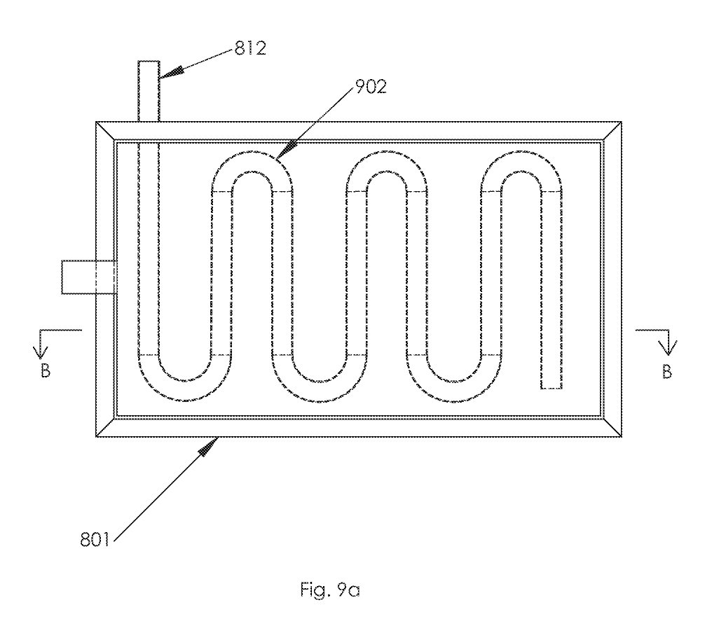

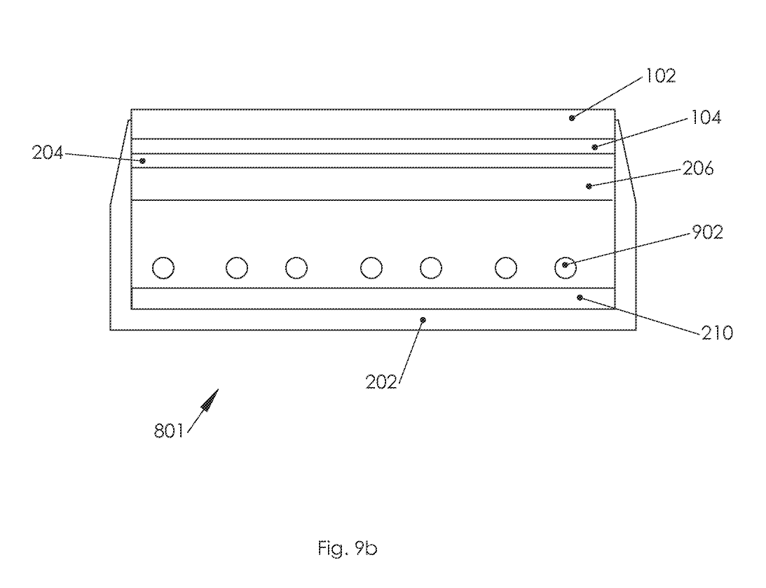

FIG. 9a illustrates a plan view of apparatus or pad 801. Apparatus 801 includes a waveguide 902 which connects to fiber 812. In the example shown in FIG. 9a, optical waveguide 902 may have a serpentine shape and be configured such that light is emitted from the sides thereof to thereby expose composite layer 102 when provided on a wound, for example. Although apparatus 801 may not include the optical sources, power supply, and control circuitry included in apparatus 100 discussed above, the remaining structure of apparatus 100 may be provided in apparatus 801, such as diffuser 206 and intermediate layer 104 and optional layer 204.

FIG. 9b illustrates a cross-sectional view of apparatus or pad 801 taken along line B-B shown in FIG. 9a.

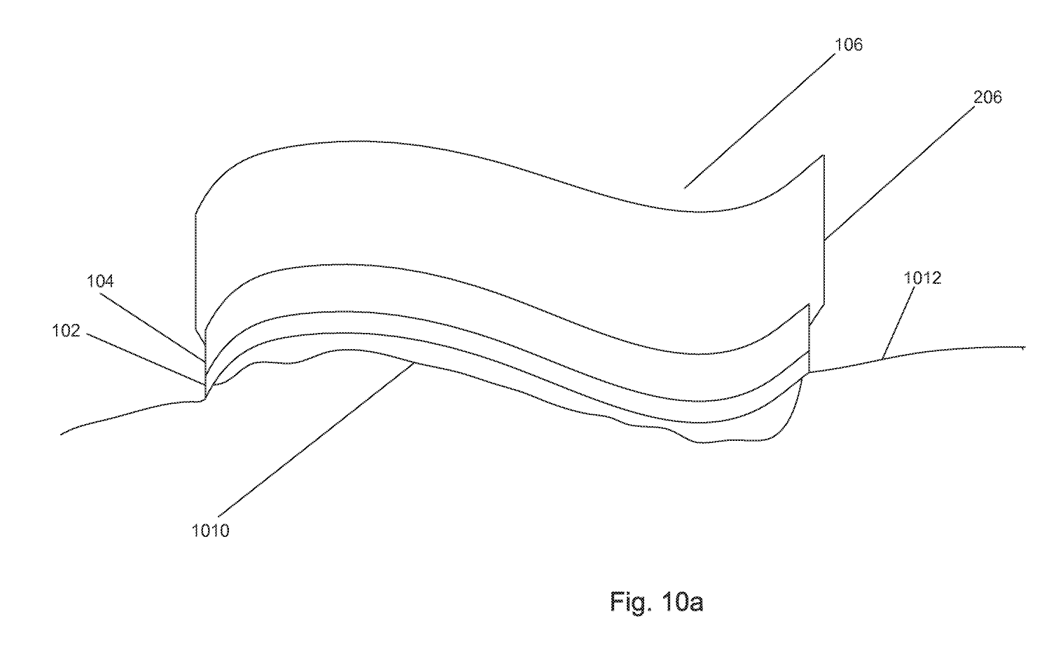

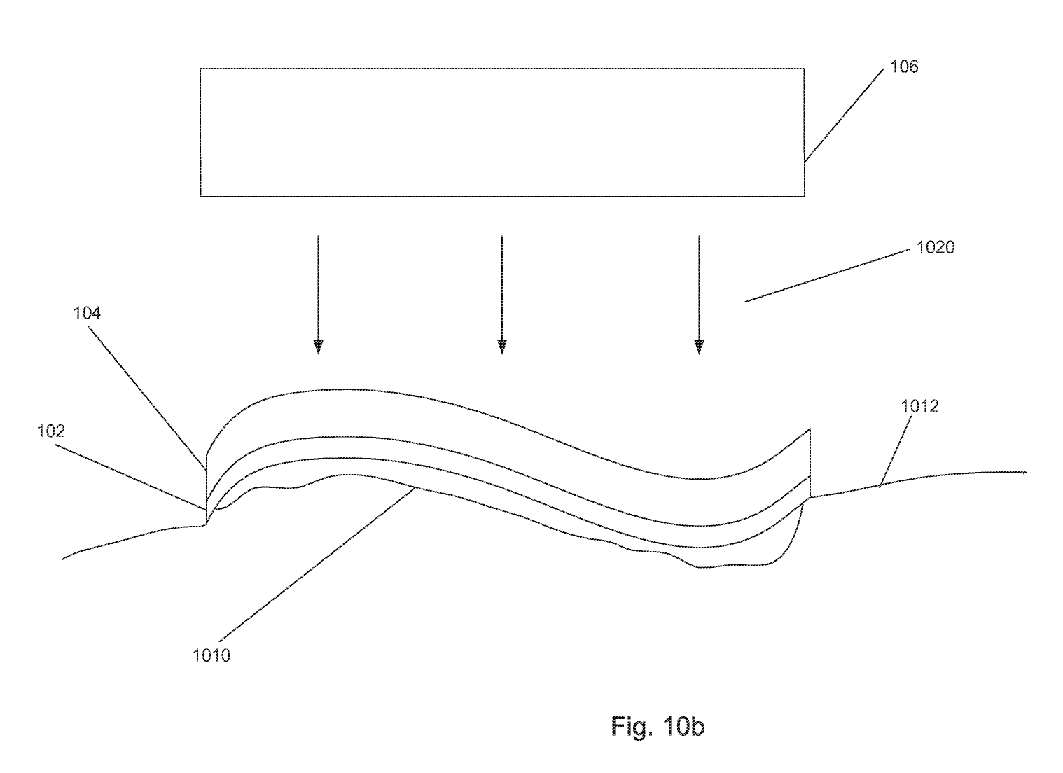

Operation of apparatus 100 and 801 will next be described with reference to FIGS. 10a, 10b, 11, and 12. As shown in FIG. 10a, composite layer 102, while still adhering to apparatus 100 is placed in contact with a diseased or infected wound 1010 of tissue 1012, such as an infected skin lesion. Preferably frame 202, intermediate layer 104 and composite layer 102 are flexible so that pad 106 and layers 102, 104 conform to the contours of tissue 1012. Optical sources 208 (if apparatus 100 is employed) may then be activated once pad 106 is appropriately placed and composite layer 102 is in contact with wound 1010. Sources 208 or waveguide 902, in turn, may emit light at a desired intensity or power for a desired duration under the control of control circuit 506, for example, in order to expose composite layer 102. As noted above, the light may have a wavelength in a range of 440 nm to 480 nm (inclusive) and may be at a wavelength of 455 nm in particular. In one example, the exposure time may be in a range of 1-30 minutes (inclusive) and the optical power may be in a range of 5 mW/cm² to 1 W/cm² (inclusive).

FIG. 10b illustrates another example in which composite layer 102 is bonded or attached to intermediate layer 104. The unattached or unbounded side of composite layer 102 may be placed in contact with wound 1010. Then pad 106, i.e., a surface of optical diffuser 206, may be brought into contact with the unbonded or unattached side of intermediate layer 104, as indicated by arrows 1020. Optical sources 208 may then be activated as noted above to generate light that passes though optical diffuser 206 and intermediate layer 104 to expose composite layer 104.

Accordingly, consistent with the present disclosure, a method is provided in which the composite layer (102) is applied to a biological tissue, e.g. wound portion or layer 1010 of tissue 1012. Then, in one example, composite layer 102 is exposed with light transmitted through intermediate layer 104, the light having a wavelength in a range of 440 nm to 480 nm, such as 455 nm.

Upon exposure to the emitted light, the chromophore in composite layer 102 may generate or provide a reactive oxygen species that destroys or kills pathogens present in wound or tissue 1010. Significant reductions in bacterial or pathogen counts have been observed consistent with the present disclosure. In one example, a bacterial count of a known MRSA bacteria was reduced 99.88% relative to a control bacterial count without the application of the composite layer and exposure of such layer.

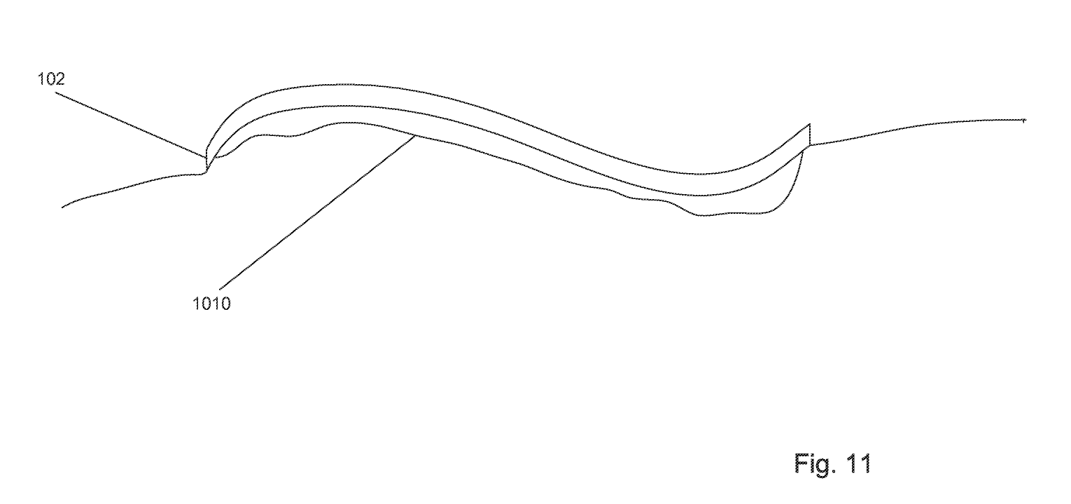

After exposure, apparatus 100 may be removed, leaving composite layer 102 on the treated wound 1010 (see FIG. 11). By providing the chromophore in composite layer 102 consistent with the present disclosure, the chromophore is maintained in a location adjacent or localized on wound 1010 and does not flow to other tissue regions. In addition, after exposure, composite layer 102 may be left on treated wound 1010.

An advantage associated with composite layer 102 is that it is believed to be non-toxic to the surrounding tissue, e.g., tissue surrounding wound 101. Conventional silver ion dressings, as noted above, however, may be toxic to the treated tissues, as well as surrounding tissue.

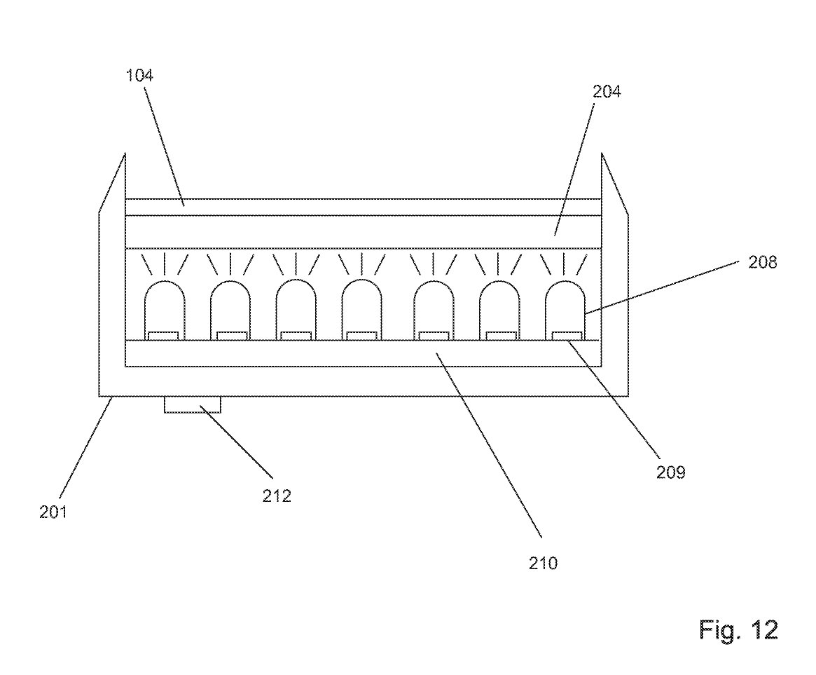

FIG. 12 illustrates apparatus 100 (also shown in FIG. 2), but without composite layer 102. In one example, apparatus 100 may be reused by providing a new, unexposed composite layer on intermediate layer 104. In the example shown in FIG. 12, intermediate layer 104 remains attached to pad 106. As noted above, however, intermediate layer 104 may not be attached to diffuser 204 of from pad 106, but may cover and be in contact with composite layer 102 during the exposure. After the exposure, intermediate layer 104 may be removed from composite layer 106.

It is understood that apparatus 801 may be applied to wound 1010 in a manner similar to that described above in connection with apparatus 100, and that apparatus 801 may similarly be removed from wound subsequent to exposure of light from waveguide 902. Also, it is understood, that apparatus 801 may be reused by providing a new, unexposed composite layer on intermediate layer 104.

Other embodiments of the invention will be apparent to those skilled in the art from consideration of the specification and practice of the invention disclosed herein. For example, a sensor may be provided in apparatus 100 or 801 to sense an amount of light absorbed by composite layer 102, for example, to determine an exposure end point. Control circuitry discussed above may be coupled or in communication with the sensor to receive a sense signal supplied by the sensor, and, in response to the sense signal, the control circuitry may control the power (voltage or current) supplied to the optical sources. For example, an appropriate sense signal may be generated by the sensor after the chromophore has completed its release of reactive oxygen, and, in response to that sense signal, the control circuitry may deactivate the optical sources.

It is intended that the specification and examples be considered as exemplary only, with a true scope and spirit of the invention being indicated by the following claims.

What is claimed is:

1. An apparatus, comprising:
a pad including a light source, the light source configured to emit light having a wavelength in a range of 440 nm-480 nm, the pad having a surface; and
a composite layer including collagen and a chromophore, the chromophore producing a reactive oxygen species upon exposure to the light, the composite layer being provided on the surface of the pad.

2. An apparatus in accordance with claim 1, wherein the pad includes an optical diffuser, the optical diffuser being configured to diffuse the light.

3. An apparatus in accordance with claim 2, wherein the optical diffuser includes glass.

4. An apparatus in accordance with claim 2, wherein the optical diffuser includes an acrylic.

5. An apparatus in accordance with claim 2, wherein the optical diffuser includes Teflon.

6. An apparatus in accordance with claim 1, further including a power supply configured to supply at least one of a voltage and a current to the light source.

7. An apparatus in accordance with claim 6, wherein the power supply is included in the pad.

8. An apparatus in accordance with claim 6, further including a circuit coupled to the power supply, the circuit being configured to regulate said at least one of the voltage and current supplied to the light source.

9. An apparatus in accordance with claim 1, wherein the pad includes a layer having polyacetic acid, a surface of the layer being the surface of the pad.

10. An apparatus in accordance with claim 1, wherein the pad includes a layer having polyvinyl alcohol, a surface of the layer being the surface of the pad.

11. An apparatus in accordance with claim 1, wherein the pad includes a layer having a plastic polymer, a surface of the layer being the surface of the pad.

12. An apparatus in accordance with claim 1, wherein the pad further includes an intermediate layer, the intermediate layer having a surface, the surface of the intermediate layer being the surface of the pad.

13. An apparatus in accordance with claim 2, further including an intermediate layer provided between the optical diffuser and the composite layer, the intermediate layer having a surface, the surface of the intermediate layer being the surface of the pad.

14. An apparatus in accordance with claim 12, wherein the intermediate layer including one of a polyacetic acid and a polyvinyl alcohol.

15. An apparatus in accordance with claim 12, wherein the intermediate layer includes a plastic polymer.

16. An apparatus in accordance with claim 1, wherein the pad includes a frame having an opening, the composite layer being provided in the opening.

17. An apparatus in accordance with claim 16, further including a reflective portion that reflects a portion of the light toward the composite layer.

18. An apparatus in accordance with claim 1, wherein the composite layer contacts the surface of the pad.

19. An apparatus in accordance with claim 1, wherein the light source includes a light emitting diode.

20. An apparatus in accordance with claim 1, wherein the light source includes a plurality of light emitting diodes.

21. An apparatus in accordance with claim 20, wherein the plurality of light emitting diodes is arranged in an array.

22. An apparatus in accordance with claim 20, wherein the pad further includes a flexible circuit substrate, the plurality of light emitting diodes being provided on the flexible circuit substrate.

23. An apparatus in accordance with claim 1, wherein a concentration of the collagen in the composite layer is 100 mg/ml (10%) up to 600 mg/ml (60%), the collagen being derivatized with a COO⁻ functional group, and the chromophore includes riboflavin.

24. An apparatus in accordance with claim 23, wherein a concentration of the riboflavin in the composite layer is within a range of 0.1% to 2.0% (w/w).

25. An apparatus in accordance with claim 1, wherein the chromophore includes riboflavin, a concentration of the riboflavin in the composite layer is substantially equal to 1.0%.

26. An apparatus in accordance with claim 1, wherein a concentration of the collagen in the composite layer is 100 mg/ml (10%) up to 600 mg/ml (60%).

27. An apparatus in accordance with claim 1, wherein the chromophore includes lumichrome.

28. An apparatus in accordance with claim 1, wherein the collagen is gelatinized.

29. An apparatus in accordance with claim 1, wherein at least a portion of the collagen is derivatized with a COO⁻ functional group.

30. An apparatus in accordance with claim 1, wherein the composite layer is detachable from the surface of the pad.

31. An apparatus, comprising:
a pad that emits light having a wavelength in a range of 440 nm-480 nm, the pad having a surface; and
a composite layer including collagen and a chromophore, the chromophore producing a reactive oxygen species upon exposure to the light.

32. An apparatus in accordance with claim 31, wherein the pad includes an optical diffuser, the optical diffuser being configured to diffuse the light.

33. An apparatus in accordance with claim 32, wherein the optical diffuser includes glass.

34. An apparatus in accordance with claim 32, wherein the optical diffuser includes an acrylic.

35. An apparatus in accordance with claim 32, wherein the optical diffuser includes Teflon.

36. An apparatus in accordance with claim 31, further including a power supply configured to supply at least one of a voltage and a current to the light source.

37. An apparatus in accordance with claim 36, wherein the power supply is included in the pad.

38. An apparatus in accordance with claim 36, further including a circuit coupled to the power supply, the circuit being configured to regulate said at least one of the voltage and current supplied to the light source.

39. An apparatus in accordance with claim 31, wherein the pad includes a layer having polylacetic acid, a surface of the layer being the surface of the pad.

40. An apparatus in accordance with claim 31, wherein the pad includes a layer having polyvinyl alcohol, a surface of the layer being the surface of the pad.

41. An apparatus in accordance with claim 31, wherein the pad includes a layer having a plastic polymer, a surface of the layer being the surface of the pad.

42. An apparatus in accordance with claim 31, wherein the pad further includes an intermediate layer, the intermediate layer having a surface, the surface of the intermediate layer being the surface of the pad.

43. An apparatus in accordance with claim 32, further including an intermediate layer provided between the optical diffuser and the composite layer, the intermediate layer having a surface, the surface of the intermediate layer being the surface of the pad.

44. An apparatus in accordance with claim 42, wherein the intermediate layer including one of a polyacetic acid and a polyvinyl alcohol.

45. An apparatus in accordance with claim 42, wherein the intermediate layer includes a plastic polymer.

46. An apparatus in accordance with claim 31, wherein the pad includes a frame having an opening, the composite layer being provided in the opening.

47. An apparatus in accordance with claim 46, further including a reflective portion that reflects a portion of the light toward the composite layer.

48. An apparatus in accordance with claim 31, wherein the composite layer contacts the surface of the pad.

49. An apparatus in accordance with claim 31, further including:
a light source; and
a waveguide, the light source supplying the light to the waveguide, such that the waveguide outputs the light to the pad.

50. An apparatus in accordance with claim 31, wherein the pad includes a port that receives the light from a light source that is external to the pad.

51. An apparatus in accordance with claim 49, wherein the pad includes at least one waveguide, the waveguide having segments, each of which supplying a corresponding portion of the light.

52. An apparatus in accordance with claim 30, wherein a concentration of the collagen in the composite layer is 100 mg/ml (10%) up to 600 mg/ml (60%), the collagen being derivatized with a COO⁻ functional group, and the chromophore includes riboflavin.

53. An apparatus in accordance with claim 52, wherein a concentration of the riboflavin in the composite layer is within a range of 0.1% to 2.0% (w/w).

54. An apparatus in accordance with claim 30, wherein the concentration of the riboflavin in the composite layer is substantially equal to 1.0%.

55. An apparatus in accordance with claim 30, wherein a concentration of the collagen in the composite layer is 100 mg/ml (10%) up to 600 mg/ml (60%).

56. An apparatus in accordance with claim 30, wherein the chromophore includes lumichrome.

57. An apparatus in accordance with claim 30, wherein the collagen is gelatinized.

58. An apparatus in accordance with claim 30, wherein at least a portion of the collagen is derivatized with a COO⁻ functional group.

59. An apparatus in accordance with claim 30, wherein the composite layer is detachable from the surface of the pad.

60. An apparatus in accordance with claim 30, wherein the composite layer is fixed to the surface of the pad.

61. A method, comprising:
applying a first layer to a biological tissue, the first layer including collagen and a chromophore, such that a first side of the first layer is in contact with the tissue, a second layer being attached to a second side of the first layer; and
exposing the first layer with light transmitted through the second layer, the light having a wavelength in a range of 440 nm to 480 nm, such that the chomrophore produces a reactive oxygen species.

62. A method in accordance with claim 61, wherein the chromophore includes riboflavin.

63. A method in accordance with claim 61, wherein a concentration of the collagen in the first layer is 100 mg/ml (10%) up to 600 mg/ml (60%), the collagen being derivatized with a COO⁻ functional group.

64. A method in accordance with claim 61, further including:
removing the second layer after said exposing, such that the first layer remains on the tissue.

65. An apparatus, comprising:
a composite layer including:
collagen, a concentration of collagen in the composite layer being 100 mg/m(10%) up to 600 mg/ml (60%), the collagen being derivatized with a COO⁻ functional group, and
riboflavin, a concentration of the riboflavin in the composite layer being a range of 0.1% to 2.0% (w/w); and
a pad, the pad having a surface, the composite layer being provided on the surface of the pad, the pad supplying light having a wavelength in a range of 440 nm to 480 nm, the pad including:
an optical source configured to output the light, and
a diffuser configured to diffuse the light, the diffuser being provided between the optical source and the composite layer.

66. An apparatus in accordance with claim 1, wherein the apparatus includes Tegaderm® provided between the composite layer and the surface of the pad.

67. An apparatus in accordance with claim 1, wherein the chromophore includes riboflavin.

68. An apparatus in accordance with claim 1, wherein the chromophore includes lumiflavin.

69. An apparatus in accordance with claim 1, wherein the light has a wavelength of 455 nm.

70. An apparatus in accordance with claim 31, wherein the apparatus includes Tegaderm® provided between the composite layer and the surface of the pad.

71. An apparatus in accordance with claim 31, wherein the chromophore includes riboflavin.

72. An apparatus in accordance with claim 31, wherein the chromophore includes lumiflavin.

73. An apparatus in accordance with claim 31, wherein the light has a wavelength of 455 nm.

74. A method in accordance with claim 61, wherein the light has a wavelength of 455 nm.

75. An apparatus, comprising:
a first layer including collagen and a chromophore, the chromophore producing a reactive oxygen species upon exposure light having a wavelength in a range of 440 nm to 480 nm, the first layer having a side; and
a second layer attached to the side of the first layer, a composition of the second layer being different than that of the first layer.

76. An apparatus in accordance with claim 75, wherein a concentration of the collagen in the first layer is 100 mg/ml (10%) up to 600 mg/ml (60%), the collagen being derivatized with a COO⁻ functional group, and the chromophore includes riboflavin.

77. An apparatus in accordance with claim 76, wherein a concentration of the riboflavin in the first layer is within a range of 0.1% to 2.0% (w/w).

78. An apparatus in accordance with claim 75, wherein the chromophore includes riboflavin, a concentration of the riboflavin in the first layer is substantially equal to 1.0%.

79. An apparatus in accordance with claim 75, wherein a concentration of the collagen in the first layer is 100 mg/ml (10%) up to 600 mg/ml (60%).

80. An apparatus in accordance with claim 75, wherein the chromophore includes lumichrome.

81. An apparatus in accordance with claim 75, wherein the chromophore includes riboflavin.

82. An apparatus in accordance with claim 75, wherein the chromophore includes lumiflavin.

83. An apparatus in accordance with claim 75, wherein the collagen is gelatinized.

84. An apparatus in accordance with claim 75, wherein at least a portion of the collagen is derivatized with a COO⁻ functional group.

85. An apparatus in accordance with claim 75, wherein the second layer includes a polymer.

86. An apparatus in accordance with claim 75, wherein the second layer includes Tegaderm®.
